(19) United States
(12) Patent Application Publication
(10) Pub. No.: US 2003/0109031 A1
(43) Pub. Date: Jun. 12, 2003

(54) System for detecting biological materials in a sample

(76) Inventors: David R. Chafin, Rochester, NY (US); Dennis M. Connolly, Rochester, NY (US)

Correspondence Address: Michael L. Goldman, NIXON PEABODY LLP, Clinton Square, P.O. Box 31051, Rochester, NY, 14603-1051, US

(21) Appl. No.: 10/288,657

(22) Filed: Nov. 5, 2002

Related U.S. Application Data
(60) Provisional application No. 60/332,838, filed on Nov. 6, 2001.

(51) Int. Cl. C12M 1/34; C12Q 1/68

(52) U.S. Cl. 435/287.2; 435/288.3; 435/6

(57) ABSTRACT

The present invention relates to a detection cartridge containing a housing defining a first chamber and a detection chip within the first chamber defined by the housing. The detection chip includes spaced apart electrical conductors fabricated on a substrate and capture probes attached to the conductors such that a gap exists between the capture probes on the spaced apart conductors. A sample, potentially containing a target molecule, can be analyzed for the presence of that target molecule by determining whether the gap has been bridged. Also disclosed is a system for detecting a target molecule in a sample which includes a detection cartridge and a support unit into which the detection cartridge can be positioned to carry out a procedure for detecting the target molecule in a sample. A method of detecting a target molecule using this system is also disclosed.

This application claims the benefit of U.S. Provisional Patent Application Serial No. 60/332,838, filed Nov. 6, 2001, which is hereby incorporated by reference in its entirety.

### FIELD OF THE INVENTION

The present invention relates to systems and methods for the detection of target molecules, such as deoxyribonucleic acids (DNA) or ribonucleic acids (RNA), from fluid samples.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification, and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics, 4:560 (1989) and the ligase chain reaction described in European Patent No. 0320308B1 to Backman et al. Transcription-based amplification methods are described in detail in U.S. Pat. No. 5,766,849 to McDonough et al., U.S. Pat. No. 5,654,142 to Kievits et al., Kwoh et al., Proc. Natl. Acad. Sci. U.S.A., 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol. 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve, 21(8):1064 (1998); Wiedbrauk et al., Journal of Clinical Microbiology, 33(10):2643-6 (1995); Deneer and Knight, Clinical Chemistry, 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology, 39(2):485-493 (2001); Al-Soud et al., Journal of Clinical Microbiology, 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092 to Fodor et al., and PCT Publication No. WO 90/15070 to Fodor et al. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection for the presence of specific DNA sequences. Most technologies related to array chips involve the coupling of a probe of known sequence to a substrate that can either be structural or conductive in nature. Structural types of array chips usually involve providing a platform where probe molecules can be constructed base by base or covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location.

However, this method requires the use of fluorescent or radioactive labels as additional materials. Such a system is expensive to use and is not amenable to being made portable for biological sample detection and identification. Furthermore, the hybridization reactions take up to two hours, which for many uses, such as detecting biological warfare agents, is simply too long. Therefore, a need exists for a system which can rapidly detect biological material in samples.

The present invention is directed to achieving these objectives.

### SUMMARY OF THE INVENTION

The present invention relates to a detection cartridge containing a housing defining a first chamber and a detection chip within the first chamber defined by the housing. The detection chip includes two or more electrically separated conductors fabricated on a substrate. Capture probes are attached to the conductors such that a gap exists between the capture probes on the electrically separated conductors. A sample, potentially containing a target molecule, can be analyzed for the presence of that target molecule by determining whether the conductors are electrically connected.

The present invention also relates to a system for detecting a target molecule in a sample. The system includes a detection cartridge that contains a housing defining a first chamber and a detection chip within the first chamber defined by the housing. The detection chip includes two or more electrically separated conductors fabricated on a substrate and capture probes attached to the conductors such that a gap exists between the capture probes on the electrically separated conductors. A sample, potentially containing a target molecule, can be analyzed for the presence of the target molecule by determining whether the conductors are electrically connected. An electrical connector extends through the housing and is coupled to the electrically separated conductors so that the presence of a target molecule connecting the capture probes on the electrically separated conductors can be detected. The system also includes a support unit with respect to which the detection cartridge can be positioned to carry out a procedure for detecting the target molecule in a sample. The support unit has an electrical coupler suitable for electrical communication with the electrical connector of the detection cartridge. As a result, the presence of the target molecule in the sample can be detected and communicated to the support unit.

Another aspect of the present invention relates to a method of detecting a target molecule. The method involves providing a detection system that includes a detection cartridge containing a housing defining a first chamber and a detection chip within the first chamber defined by the housing. The detection chip includes two or more electrically separated conductors fabricated on a substrate and capture probes attached to the conductors such that a gap exists between the capture probes on the electrically separated conductors. A sample, potentially containing a target molecule, can be analyzed for the presence of that target molecule by determining whether the conductors are electrically connected. An electrical connector extends through the housing and is coupled to the electrically separated conductors so that the presence of a target molecule connecting the capture probes on the electrically separated conductors can be detected. The system also includes a support unit with respect to which the detection cartridge can be positioned to carry out a procedure for detecting the target molecule in a sample. The support unit has an electrical coupler suitable for electrical communication with the electrical connector of the detection cartridge. A sample, potentially containing the target molecule, is injected into the first chamber of the housing. Then, the sample is processed within the first chamber under conditions effective to permit any of the target molecule present in the sample to bind to the capture probes and thereby connect the capture probes. Finally, the presence of the target molecule is detected by determining whether electricity is conducted between the electrically separated conductors.

In comparison to other detection systems which require the use of fluorescent or radioactive labels and a long reaction time, the present invention discloses a rapid and economical system for detecting target molecules in a sample. In particular, the disclosed system is amenable to being made portable for biological sample detection and identification, and is, thus, highly effective for many uses such as detecting biological warfare agents.

### BRIEF DESCRIPTION OF THE DRAWINGS

FIGS. 1A-B show a perspective view of a system for detection of a target nucleic acid molecule from a sample which includes a desk-top detection unit and a cartridge which is inserted into the desk-top unit. FIG. 1C shows a schematic view of this system.

FIG. 2A depicts a single test structure on a detection chip suitable to be positioned in first chamber 20 of the system shown in FIGS. 1A-C, where oligonucleotide probes are attached to electrical conductors in the form of spaced apart conductive fingers. FIG. 2B shows how a target nucleic acid molecule present in a sample is detected by the detection chip.

FIGS. 3A-B show a perspective view of a system for detection of a target nucleic acid molecule which includes a portable detection unit and a cartridge which is inserted into the portable unit. FIG. 3C shows a schematic view of this system.

### DETAILED DESCRIPTION OF THE INVENTION

The present invention relates to a detection cartridge containing a housing defining a first chamber and a detection chip within the first chamber defined by the housing. The detection chip includes two or more electrically separated conductors fabricated on a substrate. Capture probes are attached to the conductors such that a gap exists between the capture probes on the electrically separated conductors. A sample, potentially containing a target molecule, can be analyzed for the presence of that target molecule by determining whether the conductors are electrically connected.

The present invention also relates to a system for detecting a target molecule in a sample. The system includes a detection cartridge that contains a housing defining a first chamber and a detection chip within the first chamber defined by the housing. The detection chip includes two or more electrically separated conductors fabricated on a substrate and capture probes attached to the conductors such that a gap exists between the capture probes on the electrically separated conductors. A sample, potentially containing a target molecule, can be analyzed for the presence of the target molecule by determining whether the conductors are electrically connected. A first injection port is provided in the housing through which a sample solution can be introduced into the first chamber. An electrical connector extends through the housing and is coupled to the electrically separated conductors so that the presence of a target molecule connecting the capture probes on the electrically separated conductors can be detected. The system also includes a support unit into which the detection cartridge can be positioned to carry out a procedure for detecting the target molecule in a sample. The support unit has an electrical coupler suitable for electrical communication with the electrical connector of the detection cartridge. As a result, the presence of the target molecule in the sample can be detected and communicated to the support unit.

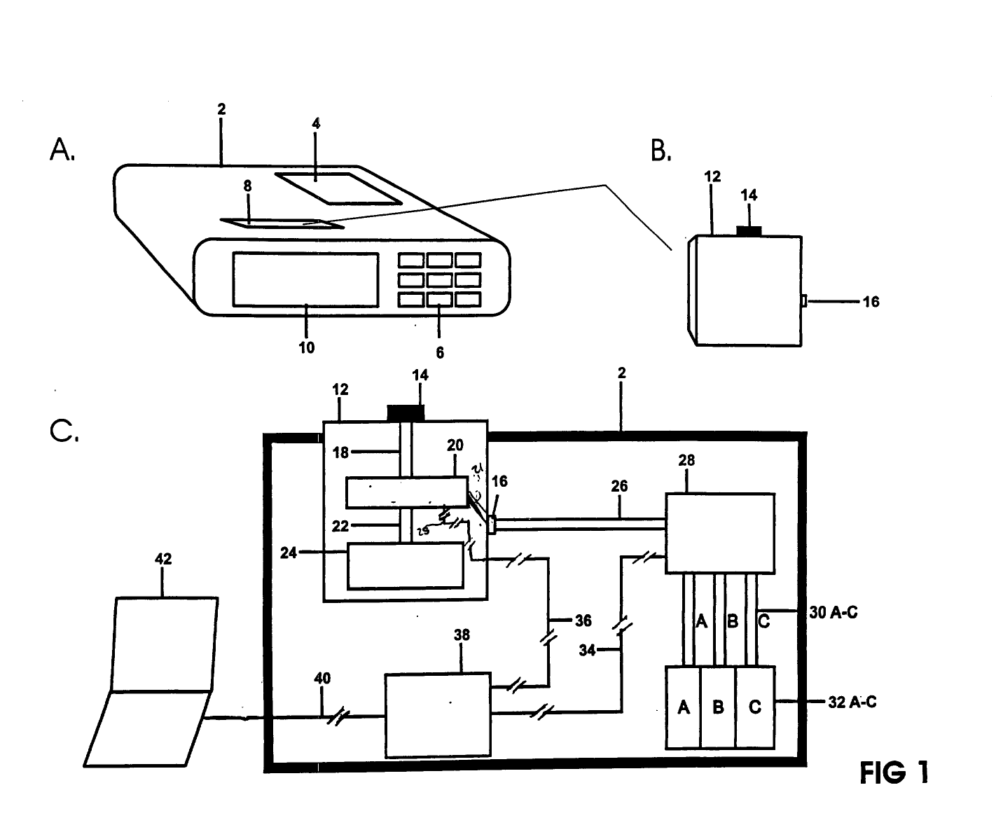

FIGS. 1A-B show a perspective view of a system for detection of a target nucleic acid molecule from a sample. This system includes a desk-top detection unit and a detection cartridge which is inserted into the desk-top unit. In this embodiment, desk-top detection unit 2 is provided with door 4 for filling reagents, control buttons 6, and visual display 10. Slot 8 in desk-top detection unit 2 is configured to receive detection cartridge 12. Detection cartridge 12 further contains first injection port 14 through which a sample solution can be introduced into a first chamber in cartridge 12 and second injection port 16 through which reagents can be introduced into the first chamber.

FIG. 1C shows a schematic view of the system utilizing desk-top detection unit 2 and cartridge 12. In this system, desk-top detection unit 2 contains containers 32A-C suitable for holding reagents and positioned to discharge the reagents into first chamber 20 of detection cartridge 12 through second injection port 16 and conduit 21. Containers 32A-C can, for example, carry a neutralizer, a buffer, a conductive ion solution, and an enhancer. The contents of these containers can be replenished through door 4. This is achieved by making containers 32A-C sealed and disposable or by making them refillable.

Pump 28 removes reagents from containers 32A-C, through tubes 30A-C, respectively, and discharges them through tube 26 and second injection port 16 into detection cartridge 12. Instead of using single pump 28 to draw reagents from containers 32A-C, a separate pump can be provided for each of containers 32A-C so that their contents can be removed individually.

Alternatively, the necessary reagents may be held in containers inside the detection cartridge. The pumps in the detection unit can force a material, such as air, water or oil, into the detection cartridge to force the reagents from the respective containers and into the first chamber. The reagents are then changed with each detection cartridge, which eliminates the buildup of salt precipitates in the detection unit.

Desk-top detection unit 12 is also provided with controller 38, which is in electrical communication with the electrical conductors of the detection cartridge 12 by means of electrical connector 36, to detect the presence of the target molecule in the sample. Controller 38 also operates pump 28 by way of electrical connector 34. Alternatively, separate controllers can be used for operating the pumps and the detection of target molecules. Digital coupling 40 permits controller 38 to communicate data to computer 42 which is external of desk-top detection unit 12.

Detection cartridge 12 contains first chamber 20 which, as noted supra, receives reagents from within desk-top detection unit 2 by way of second injection port 16 and conduit 21. A sample to be analyzed is discharged to first chamber 20 through first injection port 14 and conduit 18. As described more fully infra, the presence of a target molecule is detected in first chamber 20. Detection cartridge 12 is further provided with second chamber 24 for collecting material discharged from first chamber 20 by way of connector 22. The detection cartridge also contains electrical connector 25 extending through the housing and coupled to the electrically separated conductors in first chamber 20 so that the presence of a target molecule in a sample can be detected.

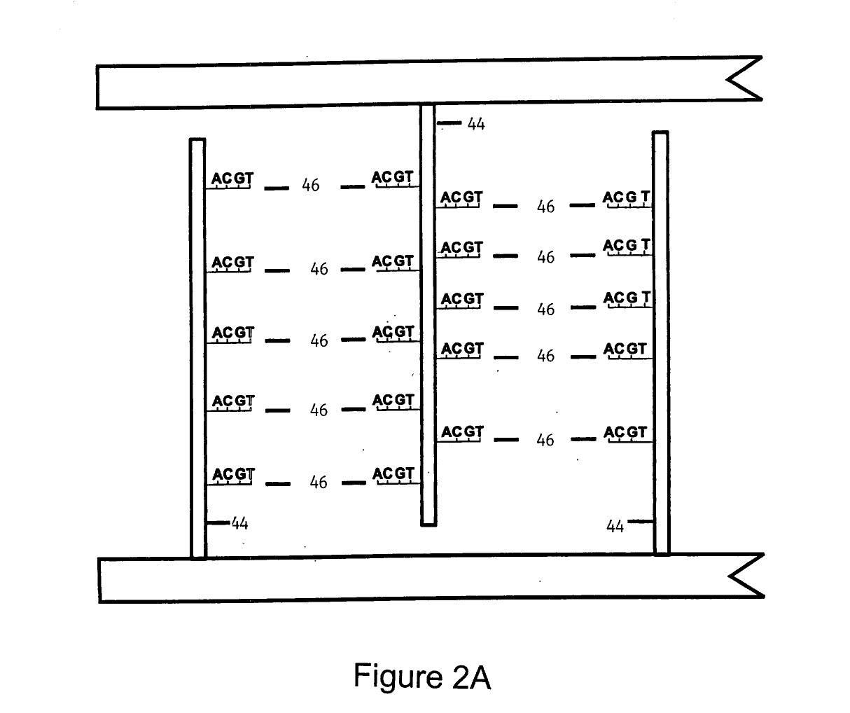

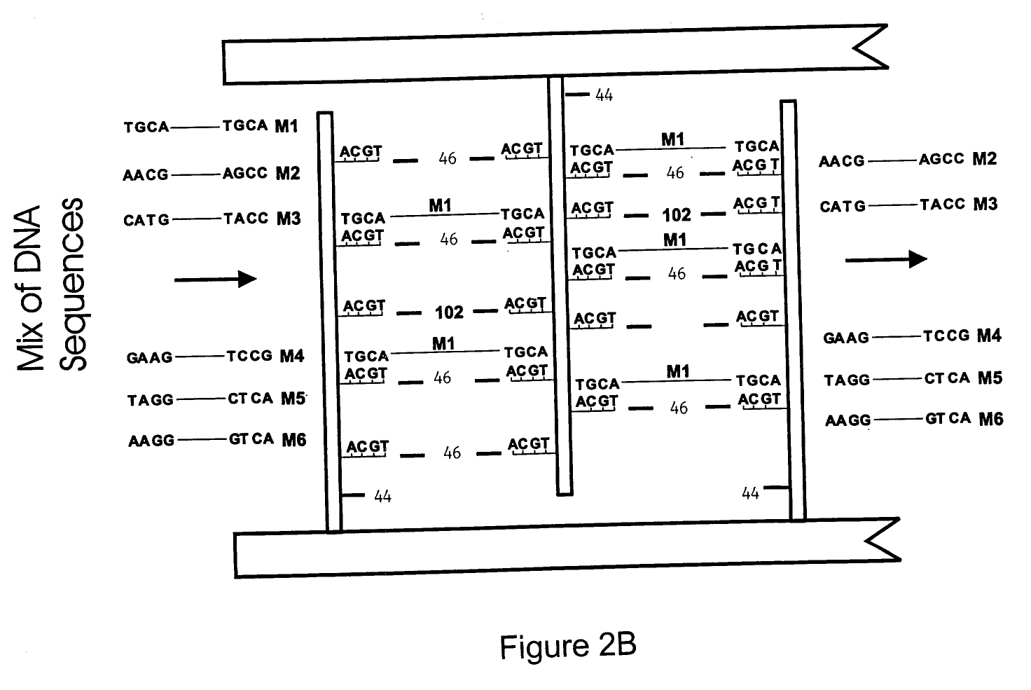

FIG. 2A depicts a single test structure on a detection chip suitable to be positioned in first chamber 20 of the system shown in FIGS. 1A-C. According to FIG. 2A, oligonucleotide probes 46 attached to spaced apart conductive fingers 44 are physically located at a distance sufficient that they cannot come into contact with one another. A sample, containing a mixture of nucleic acid molecules (i.e. M1-M6), to be tested is contacted with the fabricated device on which conductive fingers 44 are fixed, as shown in FIG. 2B. If a target nucleic acid molecule (i.e. M1) that is capable of binding to the two oligonucleotide probes is present in the sample, the target nucleic acid molecule will bind to the two probe molecules. If bound, the nucleic acid molecule can bridge the gap between the two electrodes and provide an electrical connection. Any unhybridized nucleic acid molecules (i.e. M2-M6) not captured by the probes is washed away. Here, the electrical conductivity of nucleic acid molecules is relied upon to transmit the electrical signal. Hans-Werner Fink and Christian Schoenenberger reported in Nature, 398:407-410 (1999), which is hereby incorporated by reference in its entirety, that DNA conducts electricity like a semiconductor. This flow of current can be sufficient to construct a simple switch, which will indicate whether or not a target nucleic acid molecule is present within a sample. The presence of a target molecule can be detected as an “on” switch, while a set of probes not connected by a target molecule would be an “off” switch. The information can be processed by a digital computer which correlates the status of the switch with the presence of a particular target. The information can be quickly identified to the user as indicating the presence or absence of the biological material, organism, mutation, or other target of interest. Optionally, after hybridization of the target molecules to sets of biological probes, the target molecule can be coated with a conductor, such as a metal. The coated target molecule can then conduct electricity across the gap between the pair of probes, thus producing a detectable signal indicative of the presence of a target molecule.

The detection chip, on which conductive fingers 44 are fixed, is constructed on a support. Examples of useful support materials include, e.g., glass, quartz, and silicon as well as polymeric substrates, e.g. plastics. In the case of conductive or semi-conductive supports, it will generally be desirable to include an insulating layer on the support. However, any solid support which has a non-conductive surface may be used to construct the device. The support surface need not be flat. In fact, the support may be on the walls of a chamber in a chip.

Improved methods of forming large arrays of oligonucleotides, peptides and other polymer sequences with a minimal number of synthetic steps are known. See, U.S. Pat. No. 5,143,854 to Pirrung et al. (see also, PCT Publication No. WO 90/15070 to Fodor et al.) and PCT Publication No. WO 92/10092 to Fodor et al., which are hereby incorporated by reference in their entirety, which disclose methods of forming vast arrays of peptides, oligonucleotides, and other molecules using, for example, light-directed synthesis techniques. See also, Fodor et al., Science, 251:767-77 (1991), which is hereby incorporated by reference in its entirety. These procedures for synthesis of polymer arrays are now referred to as VLSIPS™ procedures.

Methods of synthesizing desired oligonucleotide probes are known to those of skill in the art. In particular, methods of synthesizing oligonucleotides and oligonucleotide analogues can be found in, for example, Oligonucleotide Synthesis: A Practical Approach, Gait, ed., IRI Press, Oxford (1984); Kuijpers, Nucleic Acids Research, 18(17):5197 (1994); Dueholm, J. Org. Chem., 59:5767-5773 (1994); and Agrawal (ed.), Methods in Molecular Biology, 20, which are hereby incorporated by reference in their entirety. Shorter oligonucleotide probes have lower specificity for a target nucleic acid molecule, that is, there may exist in nature more than one target nucleic acid molecule with a sequence of nucleotides complementary to the oligonucleotide probe. On the other hand, longer oligonucleotide probes have decreasingly smaller probabilities of containing complementary sequences to more than one natural target nucleic acid molecule. In addition, longer oligonucleotide probes exhibit longer hybridization times than shorter oligonucleotide probes. Since analysis time is a factor in a commercial device, the shortest possible probe that is sufficiently specific to the target nucleic acid molecule is desirable. Both the speed and specificity of binding target nucleic acid molecules to oligonucleotide probes can be increased if one electrical conductor has attached a probe that is complementary to one end of the target nucleic acid molecule and the other electrical conductor has attached a probe that is complementary to the other end of the target nucleic acid. In this case, even if short oligonucleotide probes that exhibit rapid hybridization rates are used, the specificity of the target nucleic acid molecules to the two probes is high. If two different probe molecules are used, it is important that both probes are not located on the same electrical conductor, to prevent hybridization of a target nucleic acid molecule from one part of an electrical conductor to another part of the same electrical conductor. If this happens, no signal can be generated from such an attachment, and the sensitivity of the analysis is lowered.

The present invention includes chemically modified nucleic acid molecules or oligonucleotide analogues as oligonucleotide probes. An “oligonucleotide analogue” refers to a polymer with two or more monomeric subunits, wherein the subunits have some structural features in common with a naturally occurring oligonucleotide which allow it to hybridize with a naturally occurring nucleic acid in solution. For instance, structural groups are optionally added to the ribose or base of a nucleoside for incorporation into an oligonucleotide, such as a methyl or allyl group at the 2′-O position on the ribose, or a fluoro group which substitutes for the 2′-O group, or a bromo group on the ribonucleoside base. The phosphodiester linkage, or “sugar-phosphate backbone” of the oligonucleotide analogue is substituted or modified, for instance with methyl phosphonates or O-methyl phosphates. Another example of an oligonucleotide analogue includes “peptide nucleic acids” in which native or modified nucleic acid bases are attached to a polyamide backbone. Oligonucleotide analogues optionally comprise a mixture of naturally occurring nucleotides and nucleotide analogues. Oligonucleotide analogue arrays composed of oligonucleotide analogues are resistant to hydrolysis or degradation by nuclease enzymes such as RNAase A. This has the advantage of providing the array with greater longevity by rendering it resistant to enzymatic degradation. For example, analogues comprising 2′-O-methyloligoribonucleotides are resistant to RNAase A.

Many modified nucleosides, nucleotides, and various bases suitable for incorporation into nucleosides are commercially available from a variety of manufacturers, including the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill. Methods of attaching bases to sugar moieties to form nucleosides are known. See, e.g., Lukevics and Zablocka, Nucleoside Synthesis: Organosilicon Methods Ellis Horwood Limited Chichester, West Sussex, England (1991), which is hereby incorporated by reference in its entirety. Methods of phosphorylating nucleosides to form nucleotides, and of incorporating nucleotides into oligonucleotides are also known. See, e.g., Agrawal (ed), Protocols for Oligonucleotides and Analogues, Synthesis and Properties, Methods in Molecular Biology, volume 20, Humana Press, Towota, N.J. (1993), which is hereby incorporated by reference in its entirety.

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide. See U.S. patent application Ser. No. 10/159,429, which is hereby incorporated by reference in its entirety.

Another aspect of the present invention relates to a method of detecting a target molecule. The method involves providing a detection system that includes a detection cartridge containing a housing defining a first chamber and a detection chip within the first chamber defined by the housing. The detection chip includes two or more electrically separated conductors fabricated on a substrate and capture probes attached to the conductors such that a gap exists between the capture probes on the electrically separated conductors. A sample, potentially containing a target molecule, can be analyzed for the presence of that target molecule by determining whether the conductors are electrically connected. A first injection port is provided in the housing through which a sample solution can be introduced into the first chamber. An electrical connector extends through the housing and is coupled to the electrically separated conductors so that the presence of a target molecule connecting the capture probes on the electrically separated conductors can be detected. The system also includes a support unit into which the detection cartridge can be positioned to carry out a procedure for detecting the target molecule in a sample. The support unit has an electrical coupler suitable for electrical communication with the electrical connector of the detection cartridge. A sample, potentially containing the target molecule, is injected into the first chamber of the housing. Then, the sample is processed within the first chamber under conditions effective to permit any of the target molecule present in the sample to bind to the capture probes and thereby connect the capture probes. Finally, the presence of the target molecule is detected by determining whether electricity is conducted between the electrically separated conductors. The presence of the target molecule is indicated by the ability to conduct an electrical signal through the circuit. In the case where the target molecule is not present, the circuit is not be completed. Thus, the target molecule acts as a switch. The presence of a target molecule can be detected as an “on” switch, while a set of probes not connected by a target molecule would be an “off” switch. Due to the direct detection of the target molecule, the method allows for extremely sensitive detection of target molecules. The information can be processed by a digital computer which correlates the status of the switch with the presence of a particular target. The computer can also analyze the results from several switches specific for the same target, to determine specificity of binding and target concentration.

In one embodiment, the native electrical conductivity of nucleic acid molecules can be relied upon to transmit the electrical signal. Fink et al. “Electrical Conduction through DNA Molecules,” Nature, 398:407-410 (1999), which is hereby incorporated by reference in its entirety, reported that DNA conducts electricity like a semiconductor. This flow of current can be sufficient to construct a simple switch. Thus, another aspect of the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. The electrically coupled pair of oligonucleotide probes and the hybridized target nucleic acid molecule are then filled with a filling nucleic acid sequence, where the filling nucleic acid sequence is complementary to the target nucleic acid molecule and extends between the pair of oligonucleotide probes. Finally, it is determined if an electrical current can be carried between the probes, where the electrical current between the probes indicates the presence of the target nucleic acid molecule in the sample which has sequences complementary to the probes.

Alternatively, after hybridization of the target nucleic acid molecule to the oligonucleotide probes, the hybridized target nucleic acid molecule is coated with a conductive material, such as a metal, as described in U.S. Patent Application Serial No. 60/095,096 or 60/099,506, which are hereby incorporated by reference in their entirety. Examples of conductive material include silver and gold. The coated nucleic acid molecule can then conduct electricity across the gap between the pair of probes, thus producing a detectable signal indicative of the presence of a target nucleic acid molecule. Thus, the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. A conductive material is then applied over the electrically coupled pair of oligonucleotide probes and the hybridized target nucleic acid molecule. Finally, it is determined if an electrical current can be carried between the probes, where the electrical current between the probes indicates the presence of the target nucleic acid molecule in the sample which has sequences complementary to the probes.

For instance, the sodium counter ions to DNA phosphate groups can be replaced with silver ions by flooding the sample area with silver nitrate solution. After washing away excess silver nitrate, bathing the area with a photographic developer such as hydroquinone reduces the silver ions to metallic silver, which is electrically conductive. Braun et al. demonstrated that silver could be deposited along a DNA molecule (Braun et al., “DNA-Templated Assembly and Electrode Attachment of a Conducting Silver Wire,” Nature, 391:775-778 (1998), which is hereby incorporated in its entirety). A three-step process is used. First, silver is selectively localized to the DNA molecule through a Ag⁺/Na⁺ ion-exchange (Barton, Bioinorganic Chemistry eds Bertini, et al., ch. 8, University Science Books, Mill Valley, (1994), which is hereby incorporated by reference in its entirety) and complexes are formed between the silver and the DNA bases (Spiro, ed., Nucleic Acid-Metal Ion Interactions, Wiley Interscience, New York (1980); Marzeilli, et al., J. Am. Chem. Soc., 99:2797 (1977); Eichorn, ed. Inorganic Biochemistry, Vol. 2, ch 33-34, Elsevier, Amsterdam, (1973), which are hereby incorporated by reference in their entirety). The ion-exchange process may be monitored by following the quenching of the fluorescence signal of the labeled DNA. The silver ion-exchanged DNA is then reduced to form aggregates with bound to the DNA skeleton. The silver aggregates are further developed using standard procedures, such as those used in photographic chemistry (Holgate, et al., J. Histochem. Cytochem. 31:938 (1983); Birell, et al., J. Histochem. Cytochem. 34:339 (1986), which are hereby incorporated by reference in their entirety).

Thus, the detection of a target molecule using a desk-top detection system, as shown in FIGS. 1A-C, can be carried out as follows. After lysis and clarification of the sample, the sample is introduced into detection cartridge 12 through first injection port 14 and conduit 18 and into first chamber 20. Once the sample is introduced, detection cartridge 12 is inserted into slot 8 of desk-top detection unit 2 so that second injection port 16 is connected to conduit 21 and electrical connector 36 is coupled to electrical connector 25. The sample is processed in first chamber 20 containing the capture probes and electrical conductors for a period of time sufficient for detection of a target nucleic acid molecule in the sample. Processing of the sample within first chamber 20 can involve neutralizing the sample, contacting the neutralized sample with a buffer, then treating the sample with conductive ions, and treating the sample with an enhancer. Molecules that are not captured are expelled from first chamber 20 through second conduit 22 and into second chamber 24. The desk-top detection system can be programmed by a series of operation buttons 6 on the front of the device and the results can be seen on visual display 10.

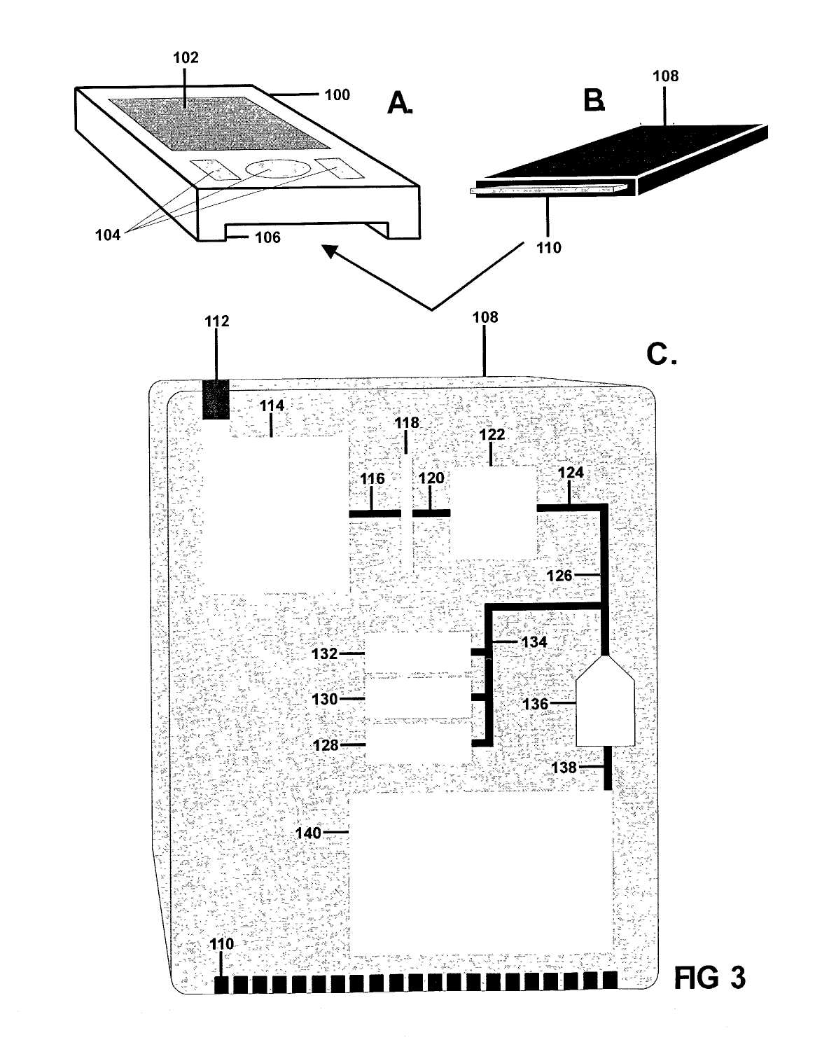

FIGS. 3A-B show a portable detection system. This system is provided with a portable unit 100 which can be in the form of a portable personal digital assistant (e.g., a Palm® unit, 3Com Corporation, Santa Clara, Calif.). Portable unit 100 is provided with visual display 102 and control buttons 104. Slot 106 is provided to receive detection cartridge 108 having electrical connector 110.

FIG. 3C shows a schematic diagram of detection cartridge 108 which is used in the portable detection system of the present invention. Detection cartridge 108 contains first injection port 112 in the housing through which a sample solution can be introduced.

Detection cartridge 108 contains a plurality of containers 128, 130, and 132 suitable for holding reagents and positioned to discharge the reagents into conduit 126 through conduit 134. Containers 128, 130, and 132 can, for example, carry a neutralizer, a buffer, and a conductive ion solution.

Sample pre-treatment chamber 114 is positioned upstream of first chamber 122, with filter 118 being positioned between pretreatment chamber 114 and first chamber 122. Conduits 116 and 120 provide a path between pretreatment chamber 114 and first chamber 122. Detection cartridge 108 also contains conduit 124 that receives material from chamber 122. Conduit 124 has a small diameter so that nucleic acid material is sheared as it passes from first chamber 122 to detection chamber 136. Detection cartridge 108 also contains a waste chamber 140 coupled to detection chamber 136 by way of conduit 138 so that material discharged from the detection chamber 136 is received in waste chamber 140. Detection cartridge 108 includes a series of electrical connectors 110 that are coupled to the electrically separated conductors in detection chamber 136, like those shown in first chamber 20 for the embodiment of FIGS. 1A-C and 2.

In operation, the detection of a target molecule using a portable detection system, as shown in FIGS. 3A-C, can be carried out as follows. After lysis and clarification of the sample, the sample solution is introduced into detection cartridge 108 through first injection port 112. Within sample pretreatment chamber 114, cells are lysed to release cellular contents. After denaturation and deprotination, the sample can be partially purified by passing it through filter 118 and depositing the solution into chamber 122. Within first chamber 138, the neutralized target nucleic acid molecule, if present in the sample, is permitted to hybridize with the capture probes on the electrically separated conductors in first chamber 136 in substantially the same way as described above with reference to FIGS. 1A-C and 2. After binding and washing, the sample is treated with a conductive ion solution from container 128, such that conductive ions are deposited on the target molecules that have hybridized to the capture probes on the detection chip. Additionally, after treatment with a conductive ion solution, the sample can be treated with an enhancer solution from container 130 to grow a continuous layer of conductive metal from the deposited conductive ions. Excess buffers and waste buffers will exit detection chamber 136 through waste tube 138 and collect in second chamber 140. The portable detection system can be programmed by operation of a series of buttons 104 on the front of portable unit 100, and the results are visualized on screen 102.

In carrying out the method of the present invention, a sample collection phase is initially carried out where bacteria, viruses or other species are collected and concentrated. The target nucleic acid molecule whose sequence is to be determined is usually isolated from a tissue sample. If the target nucleic acid molecule is genomic, the sample may be from any tissue (except exclusively red blood cells). For example, saliva, whole blood, peripheral blood lymphocytes or PBMC, skin, hair or semen are convenient sources of clinical samples. These sources are also suitable if the target is RNA. Blood and other body fluids are also a convenient source for isolating viral nucleic acids. If the target nucleic acid molecule is mRNA, the sample is obtained from a tissue in which the mRNA is expressed. If the target nucleic acid molecule in the sample is RNA, it may be reverse transcribed to DNA, but need not be converted to DNA.

A plurality of collection methods can be used depending on the type of sample to be analyzed. Liquid samples can be collected by placing a constant volume of the liquid into a lysis buffer. Airborne samples can be collected by passing air over a filter for a constant time. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

When whole cells, viruses, or other tissue samples are being analyzed, it is typically necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations such as denaturation of contaminating (DNA binding) proteins, purification, filtration, desalting, and the like.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within the extraction chamber, a separate accessible chamber, or externally introduced.

Alternatively, physical methods may be used to extract the nucleic acids and denature DNA binding proteins. U.S. Pat. No. 5,304,487 to Wilding et al., which is hereby incorporated by reference in its entirety, discusses the use of physical protrusions within microchannels or sharp edged particles within a chamber or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it is often desirable to separate the nucleic acids from other elements of the crude extract, e.g., denatured proteins, cell membrane particles, and the like. Removal of particulate matter is generally accomplished by filtration, flocculation, or the like. Ideally, the sample is concentrated by filtration, which is more rapid and does not require special reagents. A variety of filter types may be readily incorporated into the device. Samples can be forced through filters that will allow only the cellular material to pass through, trapping whole organisms and broken cell debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional chamber, or alternatively, the particular chromatographic media may be incorporated in a channel or fluid passage leading to a subsequent reaction chamber.

Alternatively, the interior surfaces of one or more fluid passages or chambers may themselves be derivatized to provide functional groups appropriate for the desired purification, e.g., charged groups, affinity binding groups and the like.

The oligonucleotide probes of the present invention may be designed to specifically recognize a variation in the sequence at the end of the probe. After the target nucleic acid molecule binds to the probes, the target nucleic acid molecule is treated with nucleases to remove the ends of the molecule which do not bind to the probes. If the confronting ends of the two probes contain sequences complementary to the target nucleic acid molecule, treatment with ligase will join the confronting ends of the two probes. The test chamber can then be heated up to denature non-ligated target nucleic acid molecule from the probes. Detection of the specific target nucleic acid molecule can then be carried out.

In a preferred embodiment of the invention, ligation methods may be used to specifically identify single base differences in sequences. Previously, methods of identifying known target sequences by probe ligation methods have been reported (U.S. Pat. No. 4,883,750 to Whiteley et al.; Wu et al., Genomics, 4:560 (1989); Landegren et al., Science, 241:1077 (1988); and Winn-Deen et al., Clin. Chem., 37:1522 (1991), which are hereby incorporated by reference in their entirety). In one approach, known as oligonucleotide ligation assay (“OLA”), two probes or probe elements which span a target region of interest are hybridized to the target region. Where the probe elements basepair with adjacent target bases, the confronting ends of the probe elements can be joined by ligation, e.g., by treatment with ligase. The ligated probe element is then assayed, evidencing the presence of the target sequence.

Hybridization assays on substrate-bound oligonucleotide arrays involve a hybridization step and a detection step. Homologous nucleotide sequences can be detected by selectively hybridizing to each other. Selectively hybridizing is used herein to mean hybridization of DNA or RNA probes from one sequence to the “homologous” sequence under stringent or non-stringent conditions (Ausubel et al., eds., Current Protocols in Molecular Biology, Vol. 1: 2.10.3, Greene Publishing Associates, Inc. and John Wiley & Sons, Inc., New York (1989), which is hereby incorporated by reference in its entirety). Hybridization and wash conditions are also exemplified in Sambrook et al., Molecular Cloning: A Laboratory Manual, Second Edition, Cold Spring Harbor, N.Y. (1989), which is hereby incorporated by reference in its entirety.

A variety of hybridization buffers are useful for the hybridization assays of the invention. Addition of small amounts of ionic detergents (such as N-lauroyl-sarkosine) are useful. LiCl is preferred to NaCl. Additional examples of hybridization conditions are provided in several sources, including: Sambrook et al., Molecular Cloning: A Laboratory Manual, 2nd Ed., Cold Spring Harbor, N.Y. (1989); Berger et al., “Guide to Molecular Cloning Techniques,” Methods in Enzymology, Volume 152, Academic Press, Inc., San Diego, Calif. (1987); and Young et al., Proc. Natl. Acad. Sci. USA, 80:1194 (1983), which are hereby incorporated by reference in their entirety. In addition to aqueous buffers, non-aqueous buffers may also be used. In particular, non-aqueous buffers which facilitate hybridization but have low electrical conductivity are preferred.

The hybridization mixture is placed in contact with the array and incubated. Contact can take place in any suitable container, for example, a dish or a cell specially designed to hold the probe array and to allow introduction of the fluid into and removal of it from the cell so as to contact the array. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, the hybridization is carried out at a temperature at or between five degrees below the melting temperature and the melting temperature. The target is incubated with the probe array for a time sufficient to allow the desired level of hybridization between the target and any complementary probes in the array. The hybridization mixture may contain an isostabilizing agent, a denaturing agent, or a renaturation accelerant.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents. Betaine (N,N,N,-trimethylglycine) can eliminate the base pair composition dependence of DNA thermal stability (Rees et al., Biochemistry, 32:137-144 (1993), which is hereby incorporated by reference in its entirety). Unlike tetramethylammonium chloride (“TMACI”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a compositions that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M. Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) Al and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 mu M to about 10 mM and, preferably, 1 mu M to about 1 mM. The CTAB buffers work well at concentrations as low as 0.1 mM.

After incubation with the hybridization mixture, the array usually is washed with the hybridization buffer, which also can include the hybridization optimizing agent. These agents can be included in the same range of amounts as for the hybridization step, or they can be eliminated altogether. Then, the array can be examined to identify the probes to which the target has hybridized.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the protease is removed or inactivated, the target nucleic acid molecule can be removed and the device is ready for use.

The number of probes may be increased in order to determine concentrations of the target nucleic acid molecule. If a plurality of each pair of oligonucleotide probes is provided, the method of the present invention can be used to identify the number of pairs of identical oligonucleotide probes between which electrical current passes to quantify the amount of the target nucleic acid molecule present in the sample. For example, several thousand repeated probes may be produced in the detection apparatus. The circuit would be able to count the number of occupied sites. Calculations could be done by the unit to determine the concentration of the target nucleic acid molecule.

The method of the present invention can be used for numerous applications, such as detection of pathogens or viruses. For example, samples may be isolated from drinking water or food and rapidly screened for infectious organisms, using probes that are complementary to the genetic material of a pathogenic bacteria. In recent times, there have been several large recalls of tainted meat products. The method of the present invention can be used for the in-process detection of pathogens in foods and the subsequent disposal of the contaminated materials. This could significantly improve food safety, prevent food borne illnesses and death, and avoid costly recalls. Detection devices with oligonucleotide probes that are complementary to the genetic material of common food borne pathogens, such as Salmonella and E. coli., could be designed for use within the food industry.

In yet another embodiment, the method of the present invention can be used for real time detection of biowarfare agents, by using probes that are complementary to the genetic material of a biowarfare agent. With the recent concerns of the use of biological weapons in a theater of war and in terrorist attacks, the device could be configured into a personal sensor for the combat soldier or into a remote sensor for advanced warnings of a biological threat. The devices which can be used to specifically identity of the agent, can be coupled with a modem to send the information to another location. Mobile devices may also include a global positioning system to provide both location and pathogen information.

In yet another embodiment, the present invention may be used to identify an individual, by using probes that are complementary to the genetic material of a human. A series of probes, of sufficient number to distinguish individuals with a high degree of reliability, are placed within the device. Various polymorphism sites are used. Preferentially, the device can determine the identity to a specificity of greater than one in 1 million, more preferred is a specificity of greater than one in one billion, even more preferred is a specificity of greater than one in ten billion. The present invention may be used to screen for mutations or polymorphisms in samples isolated from patients.

This invention may also be used for nucleic acid sequencing using hybridization techniques. Such methods are described in U.S. Pat. No. 5,837,832 to Chee et al., which is hereby incorporated by reference in its entirety.

### EXAMPLES

The following examples are provided to illustrate embodiments of the present invention but are by no means intended to limit its scope.

### Example 1

### Detection of Target Nucleic Acid Molecules in a Sample Containing Purified DNA

In a prophetic example, a 10 μl sample containing approximately 100 ng of purified DNA dissolved in hybridization buffer (100 mM NaPhosphate, pH 7.5, 0.1% SDS) with a defined length of 5.7 kilobases is injected into the denaturation chamber. The nucleic acid denatures for approximately 1 minute before the chamber is evacuated and the sample passed along to the hybridization chamber. The nucleic acid sample resides in the hybridization chamber over the test structures for 5 minutes at a temperature of 55 degrees. The sample is evacuated from the hybridization chamber with a 10 sample volume wash with hybridization buffer. The nucleic acid sample is washed into the waste chamber. A 10 sample volume wash with distilled and deionized water rinses out the chamber and prepares the sensor for chemical coating. The metallization chemistry is then mixed on a card having electrically separated conductors and passed through the hybridization chamber at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by passing a current (200 nA) through one of the two electrical test pads on each test structure and measuring the resistance between the two electrodes. Low resistance indicates the metallization process has fused two electrodes and is a positive result.

### Example 2

### Detection of Target Nucleic Acid Molecules in a Sample Containing Bacteria

In a prophetic example, a known quantity of bacteria are placed into lysis solution (Tris-CL, SDS) for 1 minute to break open bacteria. The cell debris is removed via filtration and the genomic DNA sheared by passing the solution through a point-sink shearing cartridge (65 μm diameter tubing). A 10 μl sample of the partially purified lysate in hybridization buffer (100 mM NaPhosphate, pH 7.5, 0.1% SDS) is injected into the denaturation chamber. The nucleic acid denatures for approximately 1 minute before the chamber is evacuated and the sample is passed along to the hybridization chamber. The nucleic acid sample resides in the hybridization chamber over the test structures for 5 minutes at a temperature of 55 degrees. The sample is evacuated from the hybridization chamber with a 10 sample volume wash with hybridization buffer. The nucleic acid sample is washed into the waste chamber. A 10 sample volume wash with distilled and deionized water rinses out the chamber and prepares the sensor for chemical coating. The metallization chemistry is then mixed on a card having electrically separated conductors and passed through the hybridization chamber at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by passing a current (200 nA) through one of the two electrical test pads on each test structure and measuring the resistance between the two electrodes. Low resistance indicates the metallization process has fused two electrodes and is a positive result.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A detection cartridge comprising:
a housing defining a first chamber and
a detection chip within the first chamber defined by said housing, wherein said detection chip comprises:
two or more electrically separated conductors fabricated on a substrate and
capture probes attached to the conductors such that a gap exists between the capture probes on the electrically separated conductors, wherein a sample, potentially containing a target molecule, can be analyzed for the presence of that target molecule by determining whether the conductors are electrically connected.

2. The detection cartridge of claim 1, wherein the two or more electrically separated conductors are in the form of spaced apart conductive fingers.

3. The detection cartridge of claim 2, wherein a plurality of pairs of spaced apart conductive fingers are present on the detection chip.

4. The detection cartridge of claim 1, wherein the capture probes are oligonucleotides.

5. The detection cartridge of claim 1, wherein the capture probes are peptide nucleic acid analogs.

6. The detection cartridge of claim 1 further comprising:
a first injection port in the housing through which a sample solution can be introduced into the first chamber.

7. The detection cartridge of claim 6 further comprising:
a second chamber for collecting material which has passed through the first chamber; and
a connector establishing fluid flow between the first and second chambers.

8. The detection cartridge of claim 6 further comprising:
an electrical connector extending through the housing and coupled to the electrically separated conductors so that the presence of a target molecule connecting the capture probes on the electrically separated conductors can be detected.

9. The detection cartridge of claim 8 further comprising:
a second injection port in the housing through which reagents can be introduced into the first chamber.

10. The detection cartridge of claim 8 further comprising:
a plurality of containers suitable for holding reagents and positioned to discharge the reagents into the first chamber.

11. The detection cartridge of claim 10, wherein the containers comprise:
a container carrying a neutralizer;
a container carrying a buffer;
a container carrying a conductive ion solution; and
a container carrying an enhancer.

12. The detection cartridge of claim 8 further comprising:
a sample pre-treatment chamber positioned upstream of the first chamber; and
a filter positioned between the pretreatment chamber and the first chamber.

13. The detection cartridge of claim 12 further comprising:
a pretreatment waste chamber coupled to the pretreatment chamber.

14. A system for detecting a target molecule in a sample, said system comprising:
a detection cartridge comprising:
a housing defining a first chamber;
a detection chip within the first chamber defined by said housing, wherein said detection chip comprises:
two or more electrically separated conductors fabricated on a substrate and
capture probes attached to the conductors such that a gap exists between the capture probes on the electrically separated conductors, wherein a sample, potentially containing a target molecule, can be analyzed for the presence of that target molecule by determining whether the conductors are electrically connected; and
an electrical connector extending through the housing and coupled to the electrically separated conductors so that the presence of a target molecule connecting the capture probes on the electrically separated conductors can be detected; and
a support unit with respect to which the detection cartridge can be positioned to carry out a procedure for detecting the target molecule in a sample, wherein said support unit has an electrical coupler suitable for electrical communication with the electrical connector of the detection cartridge, whereby the presence of the target molecule in the sample can be detected and communicated to the support unit.

15. The system of claim 14, wherein the detection cartridge further comprises:
a first injection port in the housing through which a sample solution can be introduced into the first chamber.

16. The system of claim 14, wherein said support unit has a slot into which the detection cartridge is inserted.

17. The system of claim 14, wherein said support unit is portable.

18. The system of claim 17, wherein said support unit is a personal digital assistant.

19. The system of claim 17, wherein the detection cartridge further comprises:
a second chamber for collecting material which has passed through the first chamber; and
a connector establishing fluid communication between the first and second chambers.

20. The system of claim 19, wherein the detection cartridge further comprises:
a sample pre-treatment chamber positioned upstream of the first chamber and
a filter positioned between the pretreatment chamber and the first chamber.

21. The system of claim 20, wherein the detection cartridge further comprises:
a pretreatment waste chamber coupled to the pretreatment chamber.

22. The system of claim 17, wherein the detection cartridge further comprises:
a plurality of containers suitable for holding reagents and positioned to discharge the reagents into the first chamber.

23. The system of claim 22, wherein the containers comprise:
a container carrying a neutralizer;
a container carrying a buffer;
a container carrying a conductive ion solution; and
a container carrying an enhancer.

24. The system of claim 14, wherein said support unit is a desk-top unit.

25. The system of claim 24, wherein the support unit further comprises:
a plurality of containers suitable for holding reagents and positioned to discharge the reagents into the first chamber of the detection cartridge through a second injection port in the housing.

26. The system of claim 25, wherein the containers comprise:
a container carrying a neutralizer;
a container carrying a buffer;
a container carrying a conductive ion solution; and
a container carrying an enhancer.

27. The system of claim 25, wherein the support unit further comprises:
a pump to remove reagents from the containers and feed them into the first chamber of the detection cartridge through the second injection port in the housing.

28. The system of claim 24, wherein the support unit further comprises:
a controller in communication with the electrical connector to enable electrical communication between the electrical conductors of the detection cartridge and the controller, whereby the presence of the target molecule in the sample can be detected and communicated to the controller.

29. The system of claim 28, wherein the support unit further comprises:
a digital coupling to permit the controller to communicate data to a computer external of the support unit.

30. The system of claim 14, wherein the electrically separated conductors are in the form of spaced apart conductive fingers.

31. The system of claim 30, wherein a plurality of pairs of spaced apart conductive fingers are present on the detection chip.

32. The system of claim 14, wherein the capture probes are oligonucleotides.

33. The system of claim 14, wherein the capture probes are peptide nucleic acid analogs.

34. A method of detecting a target molecule comprising:
providing a detection system comprising;
a detection cartridge comprising:
a housing defining a first chamber;
a detection chip within the first chamber defined by said housing, wherein said detection chip comprises:
two or more electrically separated electrical conductors fabricated on a substrate and
capture probes attached to the conductors such that a gap exists between the capture probes on the electrically separated conductors, wherein a sample, potentially containing a target molecule, can be analyzed for the presence of that target molecule by determining whether the conductors are electrically connected; and
an electrical connector extending through the housing and coupled to the electrically separated conductors so that the presence of a target molecule connecting the capture probes on the electrically separated conductors can be detected; and
a support unit with respect to which the detection cartridge can be positioned to carry out a procedure for detecting the target molecule in a sample, wherein said support unit has an electrical coupler suitable for electrical communication with the electrical connector of the detection cartridge;
injecting a sample, potentially containing the target molecule, into the first chamber of the housing;
processing the sample within the first chamber under conditions effective to permit any of the target molecule present in the sample to bind to the capture probes and thereby connect the capture probes; and
detecting the presence of the target molecule by determining whether electricity is conducted between the electrically separated conductors.

35. The method of claim 34, wherein the detection cartridge further comprises:
a first injection port in the housing through which a sample solution can be introduced into the first chamber.

36. The method of claim 34 further comprising:
inserting the detection cartridge into a slot within the support unit.

37. The method of claim 34, wherein said support unit is portable.

38. The method of claim 37, wherein said support unit is a personal digital assistant.

39. The method of claim 37 further comprising:
collecting material which has passed through the first chamber in a second chamber within the cartridge.

40. The method of claim 37 further comprising:
pre-treating the sample in a sample pre-treatment chamber positioned within the detection cartridge and upstream of the first chamber; and
retaining a portion of the pre-treated sample with a filter positioned between the pretreatment chamber and the first chamber.

41. The method of claim 37, wherein said processing comprises:
neutralizing the sample;
contacting the neutralized sample with a buffer;
treating the sample with a conductive ion solution after said contacting with a buffer; and
treating the sample with an enhancer after said treating with a conductive ion solution.

42. The method of claim 34, wherein the system is a desk-top unit.

43. The method of claim 42 wherein said processing comprises:
neutralizing the sample;
contacting the neutralized sample with a buffer;
treating the sample with a conductive ion solution after said contacting with a buffer; and
treating the sample with an enhancer after said treating with a conductive ion solution.

44. The method of claim 34, wherein the electrically separated conductors are in the form of spaced apart conductive fingers.

45. The method of claim 44, wherein a plurality of pairs of electrically separated conductive fingers are present on the detection chip.

46. The method of claim 34, wherein the capture probes are oligonucleotides.

47. The method of claim 34, wherein the capture probes are peptide nucleic acid analogs.
